(19) United States
(12) Patent Application Publication
(10) Pub. No.: US 2011/0054322 A1
(43) Pub. Date: Mar. 3, 2011

(54) Microembolic signals detection during cordiopulmonary bypass

(76) Inventor: Paolo Zanatta, Mogliano Veneto (IT)

(21) Appl. No.: 12/870,912

(22) Filed: Aug. 30, 2010

Related U.S. Application Data
(60) Provisional application No. 61/238,923, filed on Sep. 1, 2009.

(51) Int. Cl. A61B 8/14

(52) U.S. Cl. 600/443

(57) ABSTRACT

In a method of detecting microemboli in a patient, an echography probe is applied to a selected location of the patient. An ultrasonic signal is sensed with the echocardiography probe. The ultrasonic signal is processed with a processor to determine a systemic embolic load in the selected location. A representation of a systemic embolic load indicated by the ultrasonic signal is presented to a user.

### CROSS-REFERENCE TO RELATED APPLICATION(S)

This application claims the benefit of U.S. Provisional Patent Application Ser. No. 61/238,923, filed Sep. 1, 2009, the entirety of which is hereby incorporated herein by reference.

### BACKGROUND OF THE INVENTION

1. Field of the Invention

The present invention relates to cardiac surgical systems and, more specifically, to a system for detecting microembolic signals during cardiopulmonary bypass and other surgical procedures.

2. Description of the Related Art

Microembolic injuries are one of the determining factors to cognitive dysfunction after cardiac surgery as hypoperfusion, iper-rewarming and inflammation due to cardiopulmonary bypass. Transcranial Doppler (TCD) is one method used to determine the occurrence and the frequency of cerebral microembolic signals during different kinds of cardiac surgery. Thus, TCD may alert the surgical team when microemboli enter into the cerebral circulation during surgery, thus allowing preventive measures to be taken. Advances in Doppler technology have made it possible to detect not only gaseous microemboli but also the solid ones, derived from pericardial blood suction and platelets aggregation on gas microbubbles.

Unfortunately, TCD usually requires a dedicated person specifically trained in its use to be in attendance during its use. Often, cardiac surgery is performed on short notice and people trained in TCD may not be available. Therefore, either a short-notice surgery will have to be delayed, or the surgery will have to proceed without microemboli detection.

Therefore, there is a need for detecting microembolic signals indicative of a systemic embolic load, specifically derived from microbubbles, that can be used by available operating room staff.

### SUMMARY OF THE INVENTION

The disadvantages of the prior art are overcome by the present invention which, in one aspect, is a method of detecting microemboli in a patient in which an echography probe is applied to a selected location of the patient. An ultrasonic signal is sensed with the echocardiography probe. The ultrasonic signal is processed with a processor to determine a systemic embolic load in the selected location. A representation of a systemic embolic load indicated by the ultrasonic signal is presented to a user.

In another aspect, the invention is a method of detecting microemboli in a patient in which a transaesophageal echocardiography probe is applied to a selected esophageal location in the patient that is adjacent to a esophageal descending aorta. An ultrasonic signal is sensed with the transaesophageal echocardiography probe. A pattern characteristic of a microembolic signal in the ultrasonic signal is detected with a digital processor. A representation of the microembolic signal that indicates a systemic embolic load is generated. The representation of the microembolic signal is presented to a user.

In yet another aspect, the invention is a device for detecting systemic embolic load in a patient that includes an echography probe, a processor and an output device. The echography probe is configured to be placed at a predetermined location of the patient. The processor is responsive to the echography probe and is programmed to run emboli detection software that detects emboli in a signal received from the echography probe. The processor is also programmed to generate an output signal indicative of an amount of emboli detected in the signal received from the echography probe. The output device is responsive to the output signal and is configured to indicate to a user a quantitative indication of emboli detected in the patient.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1A is a schematic diagram showing application of a TEE probe to the aorta of a patient.

FIG. 1B is a schematic diagram showing a detail of application of a TEE probe to the aorta of a patient.

FIG. 2 is a schematic diagram showing one example of a TEE display.

FIG. 3 is a flowchart of one method that can be employed to detect microbubbles.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

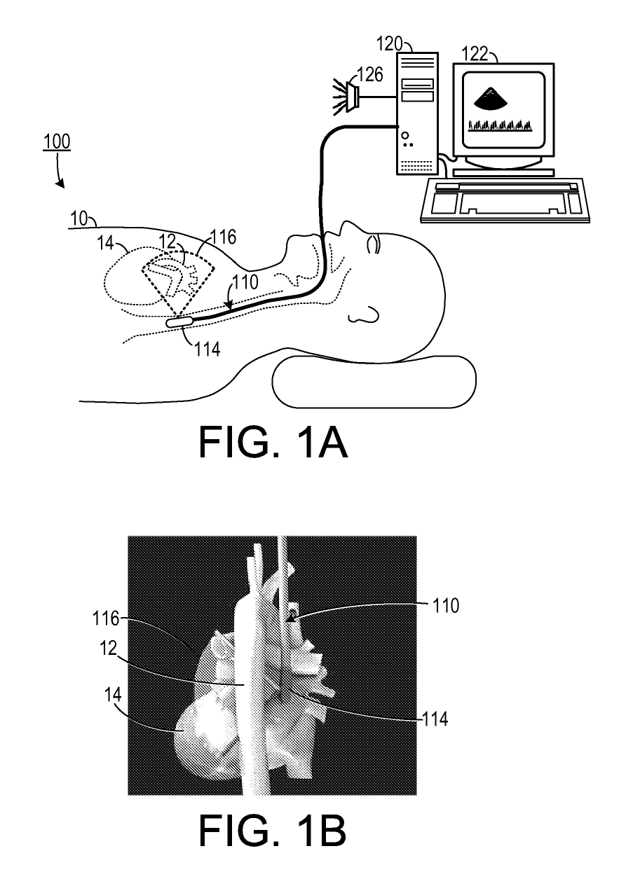

As shown in FIGS. 1A-1B, in one embodiment, one method of detecting microembolic signals in the aorta 12 of a patient 10 during heart surgery includes inserting, through the mouth of the patient 10, a transoesophageal echo device (TEE) 110 having in the distal portion an ultrasound transducer 114. The TEE device 110 is configured to perform a bidimensional or tridimensional evaluation and echocolor Doppler of the heart and the aorta. The TEE device 110 is coupled to a digital processor 120 that drives a display 122 (such as a computer video monitor) used to display processed data from the TEE device 110 (via a processor 120) such as an indication indicate to a user of a quantitative indication of emboli detected in the patient. An audio output system 126 (including, for example, a sound card and a speaker) may also be used to generate an audio representation of the output from the TEE device 110.

In one embodiment, the TEE device 110 is inserted until the echocardiogram probe 114 is adjacent the aorta 12. The TEE device 110 is then manipulated until the aorta 116 is in detecting range of the probe 114, typically in the mid oesophageal descending aortic short and long axis view. The TEE device 110 is secured relative to the patient 10. Then a pulse wave Doppler spectrum velocity is obtained according to the alignment of the thoracic aortic blood flow. The manipulation of the TEE device 110 could include showing the heart 14 on the display 122 according to the standards TEE echo views and then turning the TEE device 110 to visualize the descending thoracic aorta 116.

The digital processor 120 is programmed to run emboli detection and differentiation software (which in one clinical embodiment employed DWL® emboli detection software available from Compumedics Germany GmbH, Josef-Schüttler-Straβe 2, D-78224 Singen, Germany and Compumedics USA Ltd, 6605 West WT Harris Blvd, Suite F, Charlotte N.C. 28269, USA) that detects emboli in a signal received from the echography probe 114. The processor 120 is also programmed to generate an output signal indicative of an amount of emboli detected in the signal received from the echography probe.

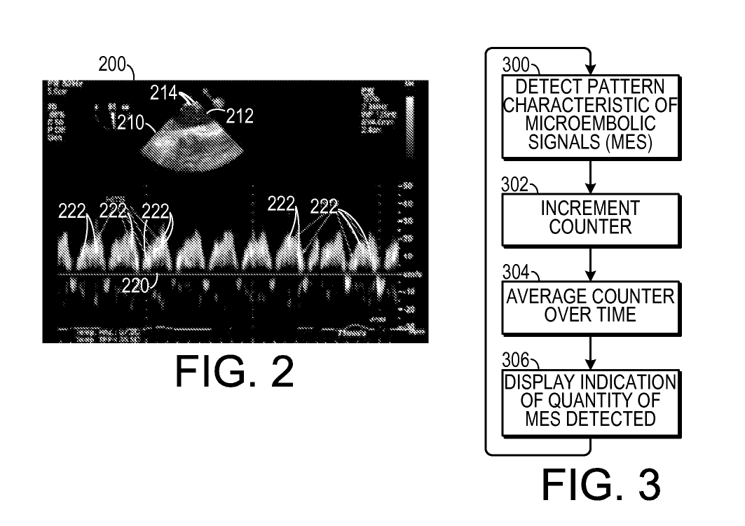

Once the surgery begins, the TEE is monitored both via a screen 200 and an audio speaker 126. The screen 200 may show the results of the TEE in several different ways. For example, as shown in FIG. 2, a current TEE image 210 may be shown. The TEE image 210 may include a region 212 that is characteristic of the aorta and microembolic signals may appear in this region 212 as spots 214 of a color or density that the area surrounding the spots 214. Similarly, the display 200 can include a continuous graphic 220 over time (pulse wave Doppler velocity spectrum of the descending thoracic aorta blood flow) in which embolic signals may appear as high intensity transient signals (HITS) 222 because of a different intensity of the ultrasound signal due to microemboli. Also, an audio representation of the TEE results can be broadcast to the user. In the audio representation, the presence of microbubbles could be represented, for example, as a discontinuous crackling sound.

In one embodiment, as shown in FIG. 3, pattern recognition software is employed to detect a pattern in the data from the TEE device that is characteristic of microembolic signals 300. Each time a microembolic signal is detected, a counter is incremented 302 and the value of the counter is averaged over time 304. As a result, an indication of the microembolic load being detected is displayed 306.

This system could give to the physician that performs the intra-operative TEE evaluation the advantage to assess the systemic embolic load during the extracorporeal circulation and also monitor the surgical, perfusionist, and anesthesiologist procedures in respect to the air contamination of cardiopulmonary bypass and others gaseous and solid source of embolization.

While in the embodiment disclosed above, the echography probe comprises a transaesophageal echocardiography probe that is placed into a selected esophageal location in the patient, such as a region adjacent to the patient's esophageal descending aorta, other types of echography probes may be used within the scope of the invention. For example, the echography probe may include a transcranial echocolor Doppler echography probe. In such an embodiment, the transcranial echocolor Doppler echography probe is temporarily fixed to a selected location on the patient's head so as to detect emboli in a Willis circle area. In another example, the echography probe could include a transthoracic echocardiography probe, which can be set for transcranial Doppler mode.

In processing the ultrasonic signal received from the probe, a pattern characteristic of a microembolic signal in the ultrasonic signal is detected using conventional pattern recognition software and a representation of the microembolic signal is then generated by the processor. The representation may be displayed as a graphic representation of the systemic embolic load, an audio representation of the systemic embolic load or both representations simultaneously.

This system gives clinicians the ability to have information about emboli in the vascular circulation during different clinical situations, such as cardiac surgery (during, for example, cardiopulmonary bypass) or out-patient procedures (for example, for the diagnosis of the patent forame ovale or other situations in which there is the need to monitor cerebral emboli).

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A method of detecting microemboli in a patient, comprising the steps of:
a. applying an echography probe to a selected location of the patient;
b. sensing an ultrasonic signal with the echocardiography probe;
c. processing the ultrasonic signal with a processor to determine a systemic embolic load in the selected location; and
d. presenting to a user a representation of a systemic embolic load indicated by the ultrasonic signal.

2. The method of claim 1, wherein the echography probe comprises a transaesophageal echocardiography probe and wherein the step of applying the echography probe comprises the step of placing the transaesophageal echocardiography probe into a selected esophageal location in the patient.

3. The method of claim 2, where the selected esophageal location comprises a region adjacent to the patient's esophageal descending aorta.

4. The method of claim 1, wherein the echography probe comprises a transcranial echocolor Doppler echography probe and wherein the step of applying an echography probe further comprises the action of temporarily fixing the transcranial echocolor Doppler echography probe to a selected location on the patient's head so as to detect emboli in a Willis circle area.

5. The method of claim 1, wherein the echography probe comprises a transthoracic echocardiography probe set for transcranial Doppler mode.

6. The method of claim 1, wherein the processing step comprises:
a. detecting a pattern characteristic of a microembolic signal in the ultrasonic signal; and
b. generating a representation of the microembolic signal.

7. The method of claim 1, wherein the presenting step comprises displaying a graphic representation of the systemic embolic load.

8. The method of claim 1, wherein the presenting step comprises generating an audio representation of the systemic embolic load.

9. A method of detecting microemboli in a patient, comprising the steps of:
a. applying a transaesophageal echocardiography probe to a selected esophageal location in the patient that is adjacent to a esophageal descending aorta;
b. sensing an ultrasonic signal with the transaesophageal echocardiography probe;
c. detecting, with a digital processor, a pattern characteristic of a microembolic signal in the ultrasonic signal;
d. generating a representation of the microembolic signal that indicates a systemic embolic load; and
e. presenting to a user the representation of the microembolic signal.

10. The method of claim 9, wherein the presenting step comprises displaying a graphic representation of the systemic embolic load.

11. The method of claim 9, wherein the presenting step comprises generating an audio representation of the systemic embolic load.

12. A device for detecting systemic embolic load in a patient, comprising:
a. an echography probe configured to be placed at a predetermined location of the patient;
b. a digital processor that is responsive to the echography probe and that is programmed to run emboli detection software that detects emboli in a signal received from the echography probe, the processor also programmed to generate an output signal indicative of an amount of emboli detected in the signal received from the echography probe; and
c. an output device that is responsive to the output signal and that is configured to indicate to a user a quantitative indication of emboli detected in the patient.

13. The device of claim 12, wherein the echography probe comprises an echocardiography probe.

14. The device of claim 13, wherein the echocardiography probe comprises a transaesophageal echocardiography probe.

15. The device of claim 12, wherein the echocardiography probe comprises a transcranial echocolor Doppler echography probe.

16. The device of claim 12, wherein the echocardiography probe comprises a transthoracic echocardiography probe.

17. The device of claim 12, wherein the output device comprises a video monitor.

18. The device of claim 12, wherein the output device comprises an audio output system.
